(19) United States
(12) Patent Application Publication
(10) Pub. No.: US 2005/0214838 A1
(43) Pub. Date: Sep. 29, 2005

(54) Methods for mapping and sequencing nucleic acids

(76) Inventors: Waclaw Szybalski, Madison, WI (US); Alfredo Villasante, Madrid (ES); Jadwiga Wild, Madison, WI (US); Maria Mendez-Lago, Madrid (ES)

Correspondence Address: QUARLES & BRADY LLP, FIRSTAR PLAZA, ONE SOUTH PINCKNEY STREET, P.O. BOX 2113 SUITE 600, MADISON, WI, 53701-2113, US

(21) Appl. No.: 11/077,530

(22) Filed: Mar. 10, 2005

Related U.S. Application Data
(60) Provisional application No. 60/551,724, filed on Mar. 10, 2004.
(60) Provisional application No. 60/551,771, filed on Mar. 11, 2004.

(51) Int. Cl. C12Q 1/68

(52) U.S. Cl. 435/6

(57) ABSTRACT

The present invention relates to methods and constructs for sequencing, mapping and ordering polynucleotide sequences. The invention finds particular applicability in analysis of repetitive DNA sequences such as heterochromatic sequences.

### CROSS-REFERENCE TO RELATED APPLICATIONS

This application claims the benefit of previously filed U.S. Provisional Patent application 60/551,724, filed Mar. 10, 2004 and previously filed U.S. Provisional Patent application 60/551,771, filed Mar. 24, 2004.

### STATEMENT REGARDING FEDERALLY SPONSORED RESEARCH OR DEVELOPMENT

Not applicable.

### BACKGROUND OF THE INVENTION

The invention relates generally to determining a nucleic acid sequence of a polynucleotide, and relates more particularly to determining a nucleic acid sequence of polynucleotides that have proven difficult to sequence, especially repetitive and highly repetitive polynucleotides.

The genome of any organism, including humans, can be divided into two components. The first component is gene-rich euchromatin, the portion of a genome in interphase. The second component is gene-poor heterochromatin, the portion of a genome that is tightly coiled up, not expressed and located near centromeres that attach the chromosomes to the mitotic spindle during somatic cell division or to the meiotic spindle during germ cell production.

Understanding the sequence and organization of an organism's genome helps researchers understand the organism's evolution and provides new insights into genetically influenced disorders. In 1990, a multinational effort to elucidate the sequence and organization of the human genome commenced. The sequencing effort, better known as the Human Genome Project (“HGP”), was divided into three phases: (1) the preliminary phase, (2) the draft phase and (3) the finishing phase.

In 2001, a first draft of the nucleotide sequence of the human genome was published by two groups. See International Human Genome Sequencing Consortium, Initial sequencing and analysis of the human genome, Nature 409:860-921 (2001); and Venter, J. C. et al., The sequence of the human genome, Science 291:1304-1351 (2001), each incorporated herein by reference as if set forth in its entirety. Neither sequence represented the complete human genome because of deficiencies in the sequencing techniques available. At least 30% of the genome was not listed in either sequence because it was refractory to the computer-assisted overlap sequencing programs. Of that 30%, nearly 10% represented a euchromatic portion refractory to sequencing of its dispersed repeats and large segmental duplications. The remainder of that 30% represented almost all of the heterochromatin which was refractory to sequencing of its highly repetitive sequences.

When the third phase of the HGP was published in 2004, 99% of the euchromatic portion had been successfully sequenced. See Stein, L., End of the beginning, Nature 431:915-916 (2004); She, X. et al., Shotgun sequence assembly and recent segmental duplications with in the human genome, Nature 431:927-930 (2004); and International Human Genome Sequencing Consortium, Finishing the euchromatic sequence of the human genome, Nature 431:931-945 (2004), each incorporated herein by reference as if set forth in its entirety. However, the heterochromatic sequences of the human genome remains to be determined—a reported 341 gaps remain in the human genome with 308 gaps reflecting the euchromatic portion and 33 gaps reflecting the heterochromatic portion. International Human Genome Sequencing Consortium, 431 Nature at 932.

The two groups that published the initial drafts of the human genome used different sequencing methods. The International Human Genome Sequencing Consortium used a bacterial artificial chromosome (“BAC”) to bacterial artificial chromosome (“BAC-to-BAC”) cloning method. BACs are cloning vectors that accept large-inserts (about 100-200 kb) more stably than cosmids or yeast artificial chromosomes (“YAC”). In the BAC-to-BAC technique, a crude physical map of the whole genome is created by cutting the chromosomes into large fragments of about 150,000 bp and then ordering the large fragments. Generating the draft sequence of the human genome with the BAC-to-BAC sequencing technique involved (1) cloning the fragments into a BAC; (2) selecting and sequencing BAC clones; and (3) assembling the ordered, sequenced BAC clones into a draft genome sequence. After this first round of sequencing, the BAC fragments were further fragmented into fragments of about 1500 bp long. These smaller fragments were cloned into a new vector, such as an M13 vector, and the process of selecting and sequencing clones was repeated with these smaller fragments.

In contrast, Celera Genomics Corporation used a whole-genome shotgun (WGS) approach. Unlike BAC-to-BAC, WGS sequencing bypassed the need to create a crude physical map and was, therefore, much faster and perhaps less accurate. Generating the draft sequence of the human genome with the WGS sequencing technique involved (1) fragmenting a genome into fragments of two size ranges, about 2000 bp and about 10,000 bp; (2) cloning each group of fragments into a plasmid; (3) selecting each group of clones to be sequenced; (4) sequencing each group of clones and (5) assembling the individual sequenced clones into an overall draft genome sequence.

Neither the BAC-to-BAC method nor the WGS method can resolve the sequence of large DNA fragments having identical or nearly identical stretches of about 500-1000 nucleotides (so-called “highly repetitive” and “moderately repetitive” fragments). Such fragments are not amenable to accurate sorting by computerized sequence alignment programs. The result in either method is spurious alignment of these DNA fragments. As a result of this shortcoming, near-complete nucleotide sequences have been determined for only three multicellular organisms—the nematode, mustard weed and the fruit fly. Inefficiency and the inability to sequence highly repetitive, or even moderately repetitive, portions of the genome demonstrate a need for innovative methods for mapping, aligning and determining the complete nucleotide sequence of an organism's genome.

### SUMMARY OF THE INVENTION

The invention relates generally to nucleic acid mapping, ordering and sequencing methods and particularly to methods that permit sequencing of genomic regions having repetitive nucleotide stretches, such as heterochromatic nucleic acid. The invention also relates to nucleic acid vectors constructed so as to facilitate the methods of the invention. The invention advantageously permits mapping of an insert cloned into a vector backbone by marking the clone with a pair of rare restriction enzyme recognition sequences that define two rare cleavage sites—a first rare cleavage site at a fixed position in the vector backbone portion and a second rare cleavage site at a random location in the insert portion. The invention further advantageously permits determination of nucleic acid sequence of a portion of a cloned insert by also providing bi-directional sequencing primer binding sites at a location in the insert that is fixed relative to the second cleavage site but which is random relative to the insert as a whole. The primer binding sites are advantageously provided near (less than about 25 nucleotides) or adjacent to the second cleavage site.

In one aspect, the present invention relates to a method for determining a nucleic acid sequence of a polynucleotide insert in a vector having a backbone portion and an insert portion containing the polynucleotide insert, where the backbone portion is marked with a first recognition sequence for a first restriction enzyme, and the polynucleotide insert is marked with a second recognition sequence for a second restriction enzyme and a pair of sequencing primer binding sites in opposing orientations, the recognition sequences for the first and second restriction enzymes defining a first- and a second cleavage site for the first- and the second restriction enzymes, respectively, the binding sites and the second cleavage site being provided at constant and defined positions relative to one another. In the method, bi-directional, primer-initiated nucleic acid sequencing reactions are performed on the vector. Sequencing proceeds bi-directionally from the primer binding sites using the tools of conventional primer-directed sequencing methods.

Relatedly, in some embodiments, suitable restriction enzymes recognize their cognate recognition sequences that define cleavage sites only rarely, e.g., each cleavage site has a cleavage frequency of less than about 1 per 10⁷ base pairs. It is noted that while some recognition sequences define a cleavage site that overlaps or is coterminus with the recognition sequences, other recognition sequences define a cleavage site spaced apart at some distance from the recognition sequences. It is further noted that the recognition sequences and cleavage sites can be defined entirely by primary sequence or by other relationship have to do with the structure or conformation of the enzyme and/or the nucleic acid substrate. The pair of recognition sequences can be identical or distinct from one another. In certain embodiments, namely where the clones are marked by interposing a nucleic acid cassette, the cassette can contain two (or more) rare recognition sequences such that the cassette can be employed more universally to mark clones having backbones that contain either of the two or more recognition sequences, whereby cleavage with a single enzyme can release a fragment of interest. In particular, suitable recognition sequences include those recognized by I-SceI and PI-SceI, as well as Achilles heel sites of the type disclosed in Koob, M. & Szybalski, W., Cleaving yeast and Escherichia coli genomes at a single site, Science 250:271-273 (1990), incorporated herein by reference as if set forth in its entirety. By way of example, if a marking cassette includes both I-SceI and PI-SceI recognition sequences, then a clone having a backbone containing either recognition sequence will be cleaved by the single enzyme that corresponds to the backbone recognition sequence.

In another aspect, the invention relates to a method for preparing a plurality of marked clones for use in the aforementioned method for determining a nucleic acid sequence of a polynucleotide insert. In the method, a nucleic acid cassette is randomly interposed into a vector having a backbone portion and an insert portion containing the polynucleotide insert, to produce a plurality of marked clones having the cassette interposed at distinct positions in the polynucleotide insert. As above, the backbone portion includes the first recognition sequence. The interposed nucleic acid cassette includes the pair of sequencing primer binding sites and the second recognition sequence, the first- and the second recognition sequences defining a first- and a second cleavage site for the first- and the second restriction enzymes, respectively, each cleavage site having a cleavage frequency of less than about 1 per 10⁷ base pairs. As above, the binding sites and the second cleavage site are provided at constant and defined positions relative to one another. In certain embodiments, the nucleic acid cassette is a transposon that integrates into the insert at a random position, and in still further embodiments, the transposon includes termini that are integratively responsive to Tn5 transposase.

In yet another aspect, the invention relates to a method for ordering a plurality of overlapping nucleic acid sequences of a cloned polynucleotide insert. Briefly, a plurality of the marked clones are obtained, optionally using the aforementioned preparing method. Bi-directional, primer-initiated nucleotide sequencing reactions are performed on the nucleic acid from the plurality of amplified clones to obtain from each marked clone bi-directional sequence data for a portion of the polynucleotide insert. The positions in the marked clones of the second cleavage site relative to the fixed, first cleavage site are mapped, e.g., by digesting marked clones with the restriction enzyme(s) to yield two DNA fragments whose sizes can be ascertained using a known method such as pulsed-field gel electrophoresis (PFGE), heteroduplex electron microscopy (EM) or optical mapping (OM), or other method that allows the precise location of the second cleavage site to be determined. EM measurements are considerably more precise than PFGE, but the method is cumbersome without automation. While automation may be available for OM, the measurements may be too imprecise because fragment size is very dependent upon hydrodynamic forces. However, when as a part of the OM procedure one cleaves the DNA fragments with restriction enzyme(s) and then automatically aligns the fragments and gaps, measurements is necessary only for the terminal fragment, nearest to the Tn5. All the other fragments, as aligned by OM, will be in common. Thus, the precision can greatly increased because the actual measured fragments will be small. Instead of cutting with restriction enzyme(s) and aligning the gaps, one could mark DNA with one or more sequence-specific agents like methyl-transferase or the oligo-RecA complexes, which could be made highly and individually visible by proper illumination and magnification. Such methods should also be amenable to automation.

Alternatively an OM map of the repetitive clone can be established using several alternative restriction enzymes, followed by selection of the enzyme that gives the most suitable restriction pattern. This can be followed by measuring the size of the SceI-SceI fragments by (1) labeling the Tn5-proximal ends by filling-in the SceI site; (2) partially digesting with the selected enzyme; (3) PAGE and Southern blotting of the products according to the principle of Smith and Birnstiel, A simple method for DNA restriction site mapping, Nucleic Acids Res. 3:2387-2398 (1976), incorporated herein by reference as if set forth in its entirety; and (4) aligning of all these partial-digest PAGE-fractionated fragments and comparison of these SceI-SceI fragments, which should establish the map, and thus the lengths of all the fragments. Consequently, this would permit one to determine the entire sequence.

Orientation of the fragment can be determined using an appropriate probe or probes from the backbone and/or insert portions. The orientation-, sequence- and mapping data are assembled and evaluated to determine an order of overlapping, oriented nucleic acid sequences of the polynucleotide insert. Sufficient information will exist to assemble the complete sequence of the heterochromatic clone, even though the individual 500-1000 heterochromatic nucleotide sequences might be identical or nearly identical clones that could not be arranged using the current sequence-overlap methods.

In certain embodiments, vector backbone portion of the marked clones is a BAC. BAC vectors suitable for carrying such inserts are disclosed in U.S. Pat. Nos. 5,874,259 and 6,472,177, each incorporated by reference herein as if set forth in its entirety. The marked clones are also advantageously amplifiable, more preferably conditionally amplifiable, such that sufficient nucleic acid from the plurality of clones can be obtained to ascertain for sequencing and mapping, and ascertaining the orientations of the fragments of each clone. See, Wild, J. et al., Conditionally amplifiable BACs: Switchingfrom single-copy to high-copy vectors and genomic clones, Genome Research 12:1434-1444 (2002); and U.S. Pat. No. 6,472,177, each incorporated by reference as if set forth herein in its entirety. It matters not whether the origin of replication is present on the vector backbone portion or in the insert portion. A suitable amplifiable origin of replication is oriV, the use of which in conjunction with a BAC vector is described in incorporated U.S. Pat. No. 6,472,177.

The disclosed ordering methods are amenable to automation using, e.g., optical mapping with labeled indexers.

### BRIEF DESCRIPTION OF THE DRAWINGS

Not applicable.

### DETAILED DESCRIPTION OF A WORKING EMBODIMENT

The present invention will be better understood upon consideration of the following non-limiting examples.

### EXAMPLES

### Example 1

(A) Cloning of DNA fragments to be sequenced in their entirety. A 100-200 kb DNA fragment, either non-repetitive (control) or repetitive (180 kb from Drosophila heterochromatic centromeric region), was cloned into a BAC vector or into pBAC/oriV, the latter permitting the amplification of DNA prior to any subsequent step, as described in Wild, J. & Szybalski, W., Copy-control pBAC/oriV vectors for genomic cloning, Methods Mol. Biol. 267:145-154 (2004); and Wild, J. & Szybalski, W., Copy-control tightly regulated expression vectors based on pBAC/oriV, Methods Mol. Bio. 267:155-167 (2004), U.S. Pat. Nos. 6,864,087 and 6,472,177, each incorporated herein by reference as if set forth in its entirety. These vectors have provided in their backbone a rare restriction enzyme recognition sequence, e.g., PI-SceI or I-SceI.

(B) Construction of a Tn5 transposon with PI-SceI and/or I-SceI very rare restriction enzyme site. The Epicentre EZ::TN™<oriV/KAN-2> system was modified by adding the very rare restriction enzyme site for PI-SceI or I-SceI, or both. These transposons also contained a selection marker (kanamycin resistance or KAN or KmR), an oriV origin of replication (requiring the TrfA replication-initiating protein), and two divergent primer binding sites.

(C) Transposon insertion library. Using an in vitro Tn5 transposition procedure (see U.S. Pat. Nos. 5,925,545; 5,948,622; 5,965,443; and 6,437,109, each incorporated herein by reference as if set forth in its entirety), BAC clones were created with the modified transposons, which were inserted on average at every 400 bp. The BAC contained two rare restriction enzyme sites, the first on the BAC backbone and the other next to the priming sites in Tn5. This arrangement allowed precise measurement of the distance between these two sites (corresponding to the priming sites and the reference point on the BAC backbone) by simply measuring the length of the SceI-SceI restriction fragments, as detailed below. After acquiring the Tn5/oriV transposons, the BAC clones became amplifiable when grown in the trfA-carrying hosts as has been described in the incorporated patents.

(D) Sequencing with primers complementary to Tn5 priming sites. Using two primers that each recognized one of the primer binding sites, two 500-nucleotide sequences were determined for each clone, using primer-directed sequencing methods known to those skilled in the art.

(E) (a) Assembly of the 500-1000-nucleotide sequences. For non-repetitive (control) DNA clones, sequences were aligned simply by ascertaining sequence overlaps between the great multitude of the 500-1000-nucleotide segments of sequence. Alternatively, the position of each 500-nucleotide fragment was determined by ascertaining the distance from the reference point of the first SceI cleavage site on the BAC backbone to the Tn5 primer site (adjacent to the second SceI cleavage site) in the insert. For repetitive DNA clones, PFGE was used to measure the length of the SceI fragments in the clones. The BioRad Gene Mapper permitted optimization of electrophoresis conditions for the specific length of the SceI fragments, which when using appropriate assembly marker sizes allows measurement of the length of SceI-SceI fragments with about 1% precision, i.e., 1 kb for the 100-kb fragments. This allowed assembly of a map, including all the 500-1000-nucleotide sequences along the BAC clone. Upon SceI-mediated cutting of a BAC clone, now with two SceI sites (one on BAC backbone and second on the inserted Tn5), two DNA bands were visible on the PFGE gels, and the size of these two has to be precisely determined. The two fragments were identified by Southern blotting using appropriate BAC-complementary probes.

(b) Tn5 orientation. Since Tn5 does insert in any of two orientations, the orientation of each transposon was determined for each BAC clone. To identify these two orientations, Southern blots with Tn5-complementary probes were performed, using the same PFGE gels and the same techniques as above. These Southern blots defined the exact structure of each Tn5-decorated clone and both 500-nucleotide sequences obtained with each of the two priming sites.

(c) Map of Tn5 priming sites. All these measurements and blotting permitted the establishment of the exact map of all transposon inserts with their Tn5 priming sites and thus in turn allowed alignment of all of the 500-nucleotide sequences along the BAC clone. The Tn5 mapping procedure permits the entire sequence of any DNA to be determined without regard to whether it contains repetitive sequences or not.

(d) Alternative methods for the length measurements and establishment of a precise map of Tn5 insertions. In addition to the PFGE, other methods are available for physical measurements of DNA length.

The present invention is not intended to be limited to the foregoing, but rather to encompass all such modifications and variations as fall within the scope of the appended claims.

1. A method for determining a nucleic acid sequence of a polynucleotide insert in a vector having a backbone portion and an insert portion, the backbone portion comprising a first recognition sequence for a first restriction enzyme, the insert portion comprising the polynucleotide insert, a second recognition sequence for a second restriction enzyme and a pair of sequencing primer binding sites in opposing orientations, the recognition sequences for the first and second restriction enzymes defining a first- and a second cleavage site for the first- and the second restriction enzymes, respectively, each cleavage site having a cleavage frequency of less than about 1 per 10⁷ base pairs, the binding sites and the second cleavage site being provided at constant and defined positions relative to one another, the method comprising the step of:
performing bi-directional, primer-initiated nucleotide sequencing reactions on the vector.

2. A method as claimed in claim 1 wherein the vector comprises an inducible origin of replication.

3. A method as claimed in claim 2 wherein the inducible origin of replication is or V.

4. A method as claimed in claim 1 wherein the vector comprises a bacterial artificial chromosome plasmid carrying the insert portion.

5. A method as claimed in claim 1 wherein the first restriction enzyme is selected from the group consisting of I-SceI and PI-SceI and the second restriction enzyme is selected from the group consisting of I-SceI and PI-SceI.

6. A method as claimed in claim 1 wherein the first and the second restriction enzymes are identical.

7. A method as claimed in claim 6 wherein the first- and the second restriction enzymes are selected from the group consisting of I-SceI and PI-SceI.

8. A method as claimed in claim 1 wherein the insert portion further comprises a nucleic acid cassette interposed into the polynucleotide insert, the cassette comprising the pair of sequencing primer binding sites and the second recognition sequence.

9. A method as claimed in claim 8 wherein the interposed cassette is a transposon.

10. A method as claimed in claim 9 wherein the transposon comprises termini integratively responsive to a Tn5 transposase.

11. A method as claimed in claim 1 wherein the insert portion comprises a third recognition sequence defining a third cleavage site for a third restriction enzyme, the third cleavage site having a cleavage frequency of less than about 1 per 10⁷ base pairs.

12. A method as claimed in claim 11 wherein the first restriction enzyme is identical to one of the second- and the third restriction enzymes.

13. A method as claimed in claim 12 wherein the first restriction enzyme is selected from the group consisting of I-SceI and PI-SceI, the second restriction enzyme is I-SceI and the third restriction enzyme is PI-SceI.

14. A method as claimed in claim 11 wherein the insert portion further comprises a nucleic acid cassette interposed into the polynucleotide insert, the cassette comprising the pair of sequencing primer binding sites and the second- and the third recognition sequences.

15. A method as claimed in claim 1 wherein the polynucleotide insert comprises repetitive DNA.

16. A method for preparing a plurality of marked clones for use in a method for determining a nucleic acid sequence of a polynucleotide insert, the method comprising the step of:
randomly interposing a nucleic acid cassette into a vector having a backbone portion and an insert portion that comprises the polynucleotide insert, to produce a plurality of marked clones having the cassette interposed at distinct positions in the polynucleotide insert,
wherein the backbone portion comprises a first recognition sequence for a first restriction enzyme, the nucleic acid cassette comprises a pair of sequencing primer binding sites in opposing orientations and a second recognition sequence for a second restriction enzyme, the first- and the second recognition sequences defining a first- and a second cleavage site for the first- and the second restriction enzymes, respectively, each cleavage site having a cleavage frequency of less than about 1 per 10⁷ base pairs, the binding sites and the second cleavage site being provided at constant and defined positions relative to one another.

17. A method as claimed in claim 16 wherein the vector comprises an inducible origin of replication.

18. A method as claimed in claim 17 wherein the inducible origin of replication is or V.

19. A method as claimed in claim 16 wherein the vector comprises a bacterial artificial chromosome plasmid carrying the insert portion.

20. A method as claimed in claim 16 wherein the first restriction enzyme is selected from the group consisting of I-SceI and PI-SceI and the second restriction enzyme is selected from the group consisting of I-SceI and PI-SceI.

21. A method as claimed in claim 16 wherein the first and the second restriction enzymes are identical.

22. A method as claimed in claim 21 wherein the first- and the second restriction enzymes are selected from the group consisting of I-SceI and PI-SceI.

23. A method as claimed in claim 16 wherein the interposed cassette is a transposon that comprises the sequencing primer binding sites and the second recognition sequence.

24. A method as claimed in claim 23 wherein the transposon comprises termini integratively responsive to a Tn5 transposase.

25. A method as claimed in claim 16 wherein the interposed cassette comprises a third recognition sequence defining a third cleavage site for a third restriction enzyme, the third cleavage site having a cleavage frequency of less than about 1 per 10⁷ base pairs, the binding sites and the second- and the third cleavage sites being provided at constant and defined positions relative to one another.

26. A method as claimed in claim 25 wherein the first restriction enzyme is identical to one of the second- and the third restriction enzymes.

27. A method as claimed in claim 26 wherein the first restriction enzyme is selected from the group consisting of I-SceI and PI-SceI, the second restriction enzyme is I-SceI and the third restriction enzyme is PI-SceI.

28. A method as claimed in claim 16 wherein the polynucleotide insert comprises repetitive DNA.

29. A method for ordering a plurality of overlapping nucleic acid sequences of a polynucleotide insert in a conditionally amplifiable vector comprising a backbone portion that comprises a first recognition sequence for a first restriction enzyme that defines a first cleavage site at a constant and defined position in the vector and an insert portion that comprises the polynucleotide insert, the method comprising the steps of:
separately amplifying a plurality of marked clones of the vector, individual marked clones comprising at an insertion site in the insert portion a pair of sequencing primer binding sites in opposing orientations and a second recognition sequence for a second restriction enzyme, the second recognition sequence defining a second cleavage site for the second restriction enzyme, each cleavage site having a cleavage frequency of less than about 1 per 10⁷ base pairs, the binding sites and the second cleavage site being provided at constant and defined orientations relative to one another;
obtaining nucleic acid from the plurality of amplified clones;
separately ascertaining the orientations of the binding sites in the nucleic acid from the plurality of amplified clones;
separately performing bi-directional, primer-initiated nucleotide sequencing reactions on the nucleic acid from the plurality of amplified clones to obtain from each marked clone bi-directional sequence data for a portion of the polynucleotide insert;
separately ascertaining the position of the second cleavage site relative to the first cleavage site in the nucleic acid to obtain mapping data for each marked clone;
evaluating the orientation-, sequence- and mapping data to determine an order of overlapping, oriented nucleotide sequences of the polynucleotide insert.

30. A method as claimed in claim 29 wherein the vector comprises an inducible origin of replication, the clone-amplifying step comprising the steps of:
providing the clone in a host cell that supports inducible amplification of the clone; and
exposing the clone-containing host cell to an amplification-inducing agent.

31. A method as claimed in claim 30 wherein the inducible origin of replication is oriV.

32. A method as claimed in claim 29 wherein the vector comprises a bacterial artificial chromosome plasmid carrying the insert portion.

33. A method as claimed in claim 29 wherein the first restriction enzyme is selected from the group consisting of I-SceI and PI-SceI and the second restriction enzyme is selected from the group consisting of I-SceI and PI-SceI.

34. A method as claimed in claim 29 wherein the first and the second restriction enzymes are identical.

35. A method as claimed in claim 29 wherein the marked clones are produced by a method comprising the steps of:
randomly interposing a nucleic acid cassette into the vector, the nucleic acid cassette comprising the binding sites and the second recognition sequence.

36. A method as claimed in claim 35 wherein the interposed cassette is a transposon that comprises the sequencing primer binding sites and the second recognition sequence.

37. A method as claimed in claim 36 wherein the transposon comprises termini integratively responsive to a Tn5 transposase.

38. A method as claimed in claim 29 wherein the insert portion further comprises a third recognition sequence defining a third cleavage site for a third restriction enzyme, the third cleavage site having a cleavage frequency of less than about 1 per 10⁷ base pairs, the binding sites and the second- and the third cleavage sites being provided at constant and defined positions relative to one another, wherein the position-ascertaining step comprises the step of separately ascertaining the position of at least one of the second- and the third cleavage sites relative to the first cleavage site in the nucleic acid to obtain mapping data for each marked clone.

39. A method as claimed in claim 38 wherein the marked clones are produced by a method comprising the steps of:
randomly interposing a nucleic acid cassette into the vector, the nucleic acid cassette comprising the binding sites, and the second- and the third recognition sequences.

40. A method as claimed in claim 39 wherein the interposed cassette is a transposon that comprises the sequencing primer binding sites and the second- and the third recognition sequences.

41. A method as claimed in claim 40 wherein the transposon comprises termini integratively responsive to a Tn5 transposase.

42. A method as claimed in claim 38 wherein the first restriction enzyme is identical to one of the second- and the third restriction enzymes.

43. A method as claimed in claim 42 wherein the first restriction enzyme is selected from the group consisting of I-SceI and PI-SceI, the second restriction enzyme is I-SceI and the third restriction enzyme is PI-SceI.

44. A method as claimed in claim 29 wherein the polynucleotide insert comprises repetitive DNA.

45. A vector comprising:
a backbone portion comprising a first recognition sequence for a first restriction enzyme; and
an insert portion comprising a polynucleotide insert, a second recognition sequence for a second restriction enzyme and a pair of sequencing primer binding sites in opposing orientations, the recognition sequences for the first and second restriction enzymes defining a first- and a second cleavage site for the first- and the second restriction enzymes, respectively, each cleavage site having a cleavage frequency of less than about 1 per 10⁷ base pairs, the binding sites and the second cleavage site being provided at constant and defined positions relative to one another.

46. A vector as claimed in claim 45 further comprising an inducible origin of replication.

47. A vector as claimed in claim 46 wherein the inducible origin of replication is or V.

48. A vector as claimed in claim 45 wherein the vector comprises a bacterial artificial chromosome plasmid carrying the insert portion.

49. A vector as claimed in claim 45 wherein the first restriction enzyme is selected from the group consisting of I-SceI and PI-SceI and the second restriction enzyme is selected from the group consisting of I-SceI and PI-SceI.

50. A vector as claimed in claim 45 wherein the first and the second restriction enzymes are identical.

51. A vector as claimed in claim 45 wherein the insert portion further comprises a nucleic acid cassette interposed into the polynucleotide insert, the cassette comprising the pair of sequencing primer binding sites and the second recognition sequence.

52. A vector as claimed in claim 51 wherein the interposed cassette is a transposon.

53. A vector as claimed in claim 52 wherein the transposon comprises termini integratively responsive to a Tn5 transposase.

54. A vector as claimed in claim 45 wherein the insert portion comprises a third recognition sequence defining a third cleavage site for a third restriction enzyme, the third cleavage site having a cleavage frequency of less than about 1 per 10⁷ base pairs.

55. A method as claimed in claim 54 wherein the first restriction enzyme is identical to one of the second- and the third restriction enzymes.

56. A method as claimed in claim 55 wherein the first restriction enzyme is selected from the group consisting of I-SceI and PI-SceI, the second restriction enzyme is I-SceI and the third restriction enzyme is PI-SceI.

57. A vector as claimed in claim 54 wherein the insert portion further comprises a nucleic acid cassette interposed into the polynucleotide insert, the cassette comprising the pair of sequencing primer binding sites and the second- and the third recognition sequences.

58. A vector as claimed in claim 45 wherein the polynucleotide insert comprises repetitive DNA.
